SEQUENCE LISTING (6 sequences)

SEQ ID NO: 1 - DNA, 20 bp, Mycobacteria
  misc_feature (1)..(20)
accgacgacatcgaccactt

SEQ ID NO: 2 - DNA, 19 bp, Mycobacteria
  misc_feature (1)..(19)
agccgatcagaccgatgtt

SEQ ID NO: 3 - DNA, 24 bp, Mycobacteria
  misc_feature (1)..(24)
cgtacggtcggcgagctgatccaa

SEQ ID NO: 4 - DNA, 25 bp, Mycobacteria
  misc_feature (1)..(25)
gacctccagcccggcacgctcacgt

SEQ ID NO: 5 - DNA, 25 bp, Mycobacteria
  misc_feature (1)..(25)
ggagcggatgaccacccaggacgtc

SEQ ID NO: 6 - DNA, 25 bp, Mycobacteria
  misc_feature (1)..(25)
cagcgggttgttctggtccatgaac

(19) United States
(12) Patent Application Publication
(10) Pub. No.: US 2009/0142757 A1
(43) Pub. Date: Jun. 4, 2009

(54) STRIP AND METHOD FOR DETECTING NUCLEOTIDE AMPLIFICATION PRODUCTS OF MYCOBACTERIUM TUBERCULOSIS AND NON-TUBERCULOUS MYCOBACTERIUM

(75) Inventors: George Chin-Sheng Chou, Tainan County (TW); Yiyang Sayion, Tainan County (TW); Jyh-Phen Ju, Tainan County (TW)

Correspondence Address: WPAT, PC;INTELLECTUAL PROPERTY ATTORNEYS, 2030 MAIN STREET, SUITE 1300, IRVINE, CA, 92614, US

(73) Assignee: ASIAGEN CORPORATION, Tainan County (TW)

(21) Appl. No.: 11/948,389

(22) Filed: Nov. 30, 2007

(51) Int. Cl. C12Q 1/68; C12M 1/00

(52) U.S. Cl. 435/6; 435/287.2

(57) ABSTRACT

The present invention provides a test strip and a method for rapid identifying the presence of the amplified DNA product of Mycobacterium tuberculosis and non-tuberculous mycobacteria.

### FIELD OF THE INVENTION

The present invention relates to a strip and a method for rapid identifying the presence of the amplified DNA product of Mycobacterium tuberculosis and non-tuberculous mycobacteria.

### BACKGROUND OF THE INVENTION

Mycobacterium tuberculosis is the bacterium that causes most cases of tuberculosis (abbreviated as TB for tubercle bacillus) which is a common and deadly infectious disease. The bacteria usually attack the respiratory system, but can also affect other parts of the body. TB spreads through the air when a person carrying TB in the lungs or throat coughs, sneezes or talks. In 2004, mortality and morbidity statistics included 14.6 million chronic active TB cases, 8.9 million new cases, and 1.6 million deaths, mostly in developing countries. However, a rising number of people in the developed world are contracting tuberculosis because their immune systems are compromised by immunosuppressive drugs, substance abuse or HIV/AIDS.

Nontuberculous mycobacteria (NTM) do not cause the disease we call tuberculosis. Infection with these organisms is not transmissable, therefore they do not have public health implications and diseases caused by them, unlike those caused by M. tuberculosis, are not notifiable. However, histological tissues infected by these mycobacteria appear identical to those infected by M. tuberculosos with characteristic granulomata formation leading to caseous necrosis. On direct smear, all mycobacteria stain positive to acid and alcohol fast Zehl-Neelsen and are therefore indistinguishable from one another. Only on culture do the species specific characteristics permit identification The result is that patients, with pulmonary infections caused by NTM, are usually first diagnosed as having tuberculosis with the public health implications which that brings. Diagnosis is only revised when cultures are available some six weeks later, which may result in confusion of patients and doctors alike.

The early and accurate detection of M. tuberculosis in clinical samples is taking on growing importance in the control of tuberculosis both for the clinical treatment of infected patients and for the identification of exposed individuals at risk. Molecular techniques such as the Polymerase Chain Reaction (PCR) of specific DNA of species of mycobacteria is probably one of the most promising novel approaches for rapid, specific and sensitive diagnosis.

Canadian Pat. No. 02223705 Jia Bei Zhu et al. discloses one step assay method for detecting the end product of nucleotide amplification products by labeling the probes used for the DNA amplification with an immunogenic molecule or an affinity ligand compound and detecting the end products with a test strip pre-immobilized with two kinds of antibodies and/or ligands.

### BRIEF DESCRIPTION OF THE DRAWINGS

FIG. 1 shows the concept of example design.

FIG. 2 shows the relative positions of primers.

FIG. 3 shows the effect of different ratio of TB and NTM primers.

FIG. 4 shows the effect of different running buffers. C: control band, T: TB test band, N: NTM test band, TB(+): TB positive, NTM(+): NTM positive, TB(−): TB negative, and NTM(−): NTM negative.

FIG. 5 shows the specificity of primers and the strip. TB: Mycobacterium Tuberculosis, NTM: Non-tuberculous Mycobacterium, H: Haemophilus Influenzae, S: Staphylococcus aureus, K: Klebsiella pneumoniae, and P: Streptococcus pneumonia.

FIG. 6 shows the detecting limitation of TB/NTM Duplex PCR and TB/NTM Nested Duplex PCR.

FIG. 7 shows the detecting limitation of the strip when DNA is amplified by TB/NTM Nested Duplex PCR.

### SUMMARY OF THE INVENTION

The present invention provides a test strip for detecting nucleotide amplification products of Mycobacterium Tuberculosis (TB) and Non-tuberculous Mycobacterium (NTM), comprising: (a) an Avidin-Gold release region and (b) test bands. The present invention also provides a method for detecting TB and NTM, comprising the following steps: (a) amplifying sample DNA with labeled primers; (b) mixing the amplified DNA product with running buffer; (c) dipping the test strip into the mixture; and (d) allowing the mixture to move towards the reaction region of the strip.

### DETAILED DESCRIPTION OF THE INVENTION

Tuberculosis, which is caused by Mycobacterium tuberculosis, ranks the first in mortality and morbidity among infectious diseases. However, because of the similar clinical manifestation caused by Mycobacterium tuberculosis and non-tuberculous mycobacteria, the conventional diagnostic method is not only time-consuming but confusing. Rapid diagnosis and accurate identification of Mycobacterial species can facilitate clinical treatment and reduce the risk of transmission. The strip and method disclosed in the present invention provide a fast and convenient way for Mycobacteria identification from samples prepared from patients; and can test two Mycobacteria such as TB and NTM at one strip.

The strip is a piece of water absorbent paper comprises, from left to right, an Avidin-Gold release region and a test region with test bands. A control band can also be comprised and positioned after the test region. The Avidin-Gold release region is covered with colloidal gold particles linked with Avidin. In addition, two kinds of antibodies against the immunogenic labeled primers described below are pre-immobilized on two test bands respectively, while on the control band, BSA conjugated Biotin is prefixed.

The method comprises four steps. In the first step, specially designed primers containing Biotin in one strand and an immunogenic molecule such as DIG or FITC in the other strand is used for amplifying the sample DNA prepared from patients by Polymerase Chain Reaction (PCR). The primers are targeted to a gene, such as rpoB gene, includes highly conserved regions capable of being detected in all the species of mycobacteria and yet distinguishable from each species. After amplification, the DNA product is mixed with running buffer. In the following steps, the left side of the test strip is dipped into the mixture, allowing the mixture to move from left side to the right side of the strip by capillary action.

If the target DNA is present in the sample and is amplified, the DNA product will contain Biotin at one side and immunogenic molecule at the other side. The Biotin in the DNA product will bind to Avidin at the Avidin-Gold release region by biotinylation and thus be linked with colloidal gold particle. When reaching the test region, the anti-immunogenic molecule antibody on the test band will bind to the immunogenic molecule in the DNA product. The colloidal gold particle will also be taken and bind to the control band as long as Biotin is presented in the sample and works well with Avidin-Gold in the Avidin-Gold release region, allowing Avidin-Gold to move towards the right side of the strip. Because of the red color nature of the colloidal gold, both test band and the control band will be appeared as red bands. Red band appears on the test band suggests a positive result. If the test target is not present in the mixture, i.e. no DNA is amplified, no labeled DNA is present. On the strip, no reaction will occur in the test band, and a red colored band will only appear on the control band regardless the presence of the target DNA in the sample.

In one series of embodiments, the order of region and bands on the strip from left to right is Avidin-Gold release region, TB test band, NTM test band and Control band. The TB test band is covered with pre-immobilized anti-DIG antibody, and the NTM test band is covered with pre-immobilized anti-FITC antibody. The Control band is covered with BSA-Biotin.

The primer pair targeted to TB DNA is labeled with Biotin at one strand and DIG at the other strand. The primer pair targeted to NTM DNA is labeled with Biotin at one strand and FITC at the other strand. If TB is presented in the sample, the TB DNA will be amplified and thus the TB test band and the control band will appear red. If NTM is presented in the sample, the NTM DNA will be amplified and thus the NTM test band and the control band will appear red. If both TB and NTM are presented in the sample, the TB DNA and NTM DNA will be amplified and thus the TB test band, the NTM test band and the control band will appear red. If none of TB and NTM is presented in the sample, no DNA will be amplified and thus no test band appears red, only the control band appears red.

### EXAMPLE

The examples below are non-limiting and are merely representative of various aspects and features of the present invention.

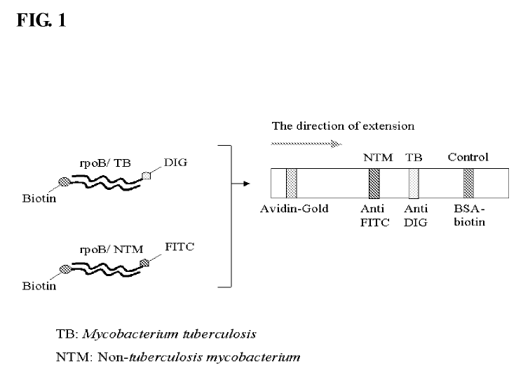

The concept of the example is shown in FIG. 1.

### Example 1

### Amplification of Target DNA

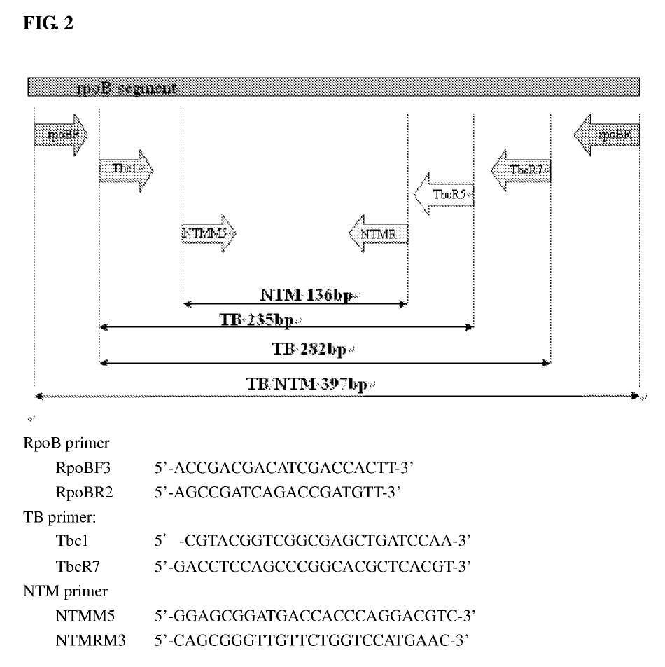

The relative position of the primers described below is shown in FIG. 2. rpoB gene of TB/NTM was first amplified by PCR using RpoB primers. The sequence of sense primer RpoBF3 is 5′-ACCGACGACATCGACCACTT-3′ as shown in SEQ ID NO: 1. The sequence of antisense primer RpoBR2 is 5′-AGCCGATCAGACCGATGTT-3′ as shown in SEQ ID NO: 2. The first PCR condition was as follows:

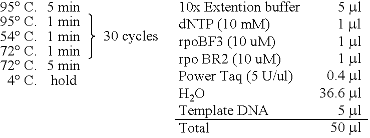

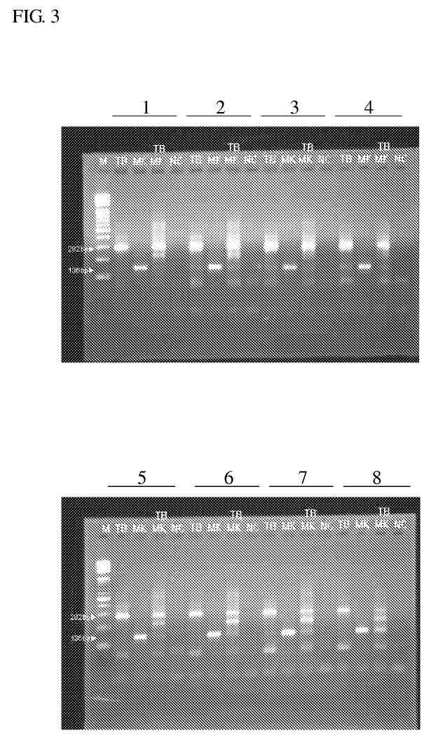

The first PCR product was then used as template for the second PCR. The primers used for the second PCR were TB primer and NTM primer. The sequence of TB sense primer Tbc1 is 5-CGTACGGTCGGCGAGCTGATCCAA-3′ as shown in SEQ ID NO: 3; and the sequence of TB antisense primer TbcR is 5′-GACCTCCAGCCCGGCACGCTCACGT-3′ as shown in SEQ ID NO: 4. The sequence of NTM sense primer NTMM5 is 5′-GGAGCGGATGACCACCCA GGACGTC-3′ as shown in SEQ ID NO: 5; and the sequence of NTM antisense primer NTMRM3 is 5′-CAGCGGGTTGTTCTGGTCCATGAAC-3′ as shown in SEQ ID NO: 6. The ratio of TB primer and NTM primer was then tested and the result showed that the best ratio was 0.3 μl TB primer plus 1 μl NTM primer (Table 1)(FIG. 3).

TABLE 1

| | 1 | 2 | 3 | 4 | 5 | 6 | 7 | 8 |
|---|---|---|---|---|---|---|---|---|
| 10xBuffer | 5 | 5 | 5 | 5 | 5 | 5 | 5 | 5 |
| dNTP | 0.5 | 0.5 | 0.5 | 0.5 | 0.5 | 0.5 | 0.5 | 0.5 |
| Tbc 1- | 0.5 | 1 | 1 | 1.5 | 0.5 | 0.3 | 0.5 | 0.3 |
| biotin | | | | | | | | |
| TbcR7-DIG | 0.5 | 1 | 1 | 1.5 | 0.5 | 0.3 | 0.5 | 0.3 |
| NTMM5- | 0.5 | 1 | 0.5 | 0.5 | 1 | 1 | 1.5 | 1.5 |
| biotin | | | | | | | | |
| NTMRM3- | 0.5 | 1 | 0.5 | 0.5 | 1 | 1 | 1.5 | 1.5 |
| FITC | | | | | | | | |
| Taq | 0.2 | 0.2 | 0.2 | 0.2 | 0.2 | 0.2 | 0.2 | 0.2 |
| DNA | 5 | 5 | 5 | 5 | 5 | 5 | 5 | 5 |
| H₂O | 37.3 | 35.3 | 36.3 | 35.3 | 36.3 | 36.7 | 35.3 | 35.7 |
| Total | 50 | 50 | 50 | 50 | 50 | 50 | 50 | 50 |

According to the test result, the second PCR condition was as follows:

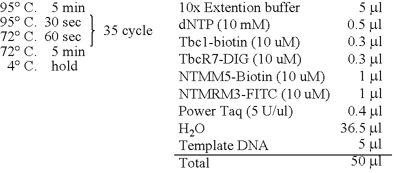

### Example 2

### Running Buffer

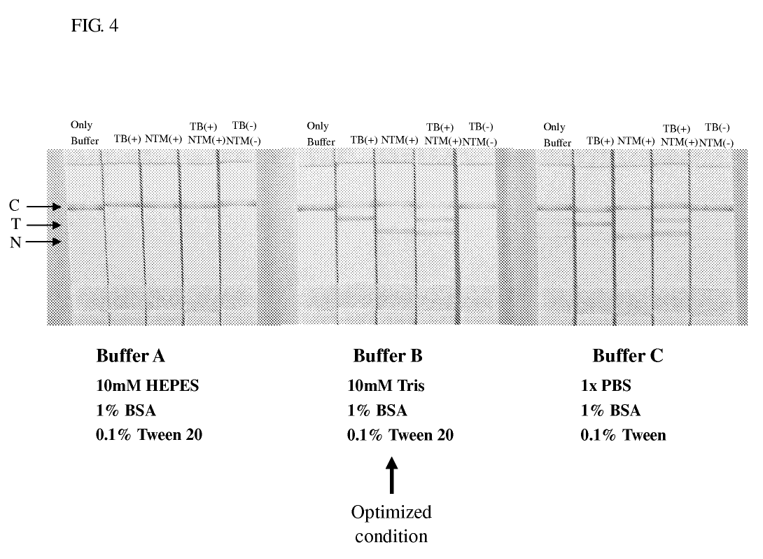

Three kinds of running buffer were tested. Buffer A contained 10 mM HEPES, 1% BSA, and 0.1% Tween 20. Buffer B contained 10 mM Tris, 1% BSA, and 0.1% Tween 20. Buffer C contained 1×PBS, 1% BSA, and 0.1% Tween 20. The result showed that Buffer B was the optimized condition (FIG. 4).

### Example 3

### The Specificity of the Primers and TB/NTM Strip

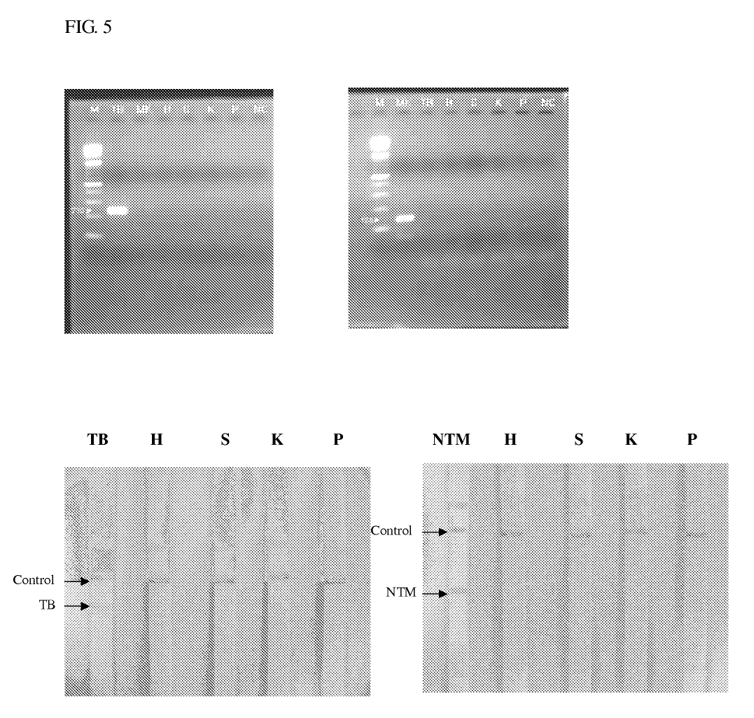

To test the specificity of the primers described above, the genome of four additional kinds of bacteria often exist in the respiratory tract were introduced to run the test steps described above. They were Haemophilus influenzae, Staphylococcus aureus, Klebsiella pneumoniae, and Streptococcus pneumonia. The result showed that the primers and the strip were specific to TB and NTM (FIG. 5).

### Example 4

### The Sensitivity of TB/NTM Strip

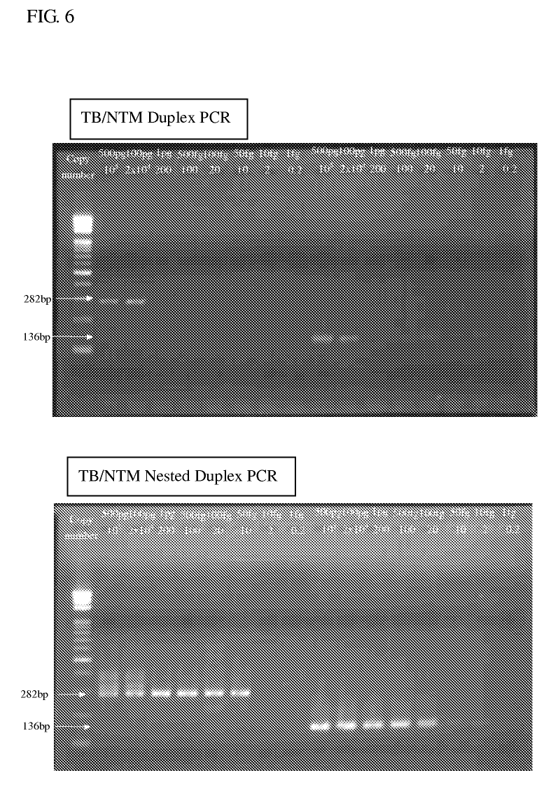

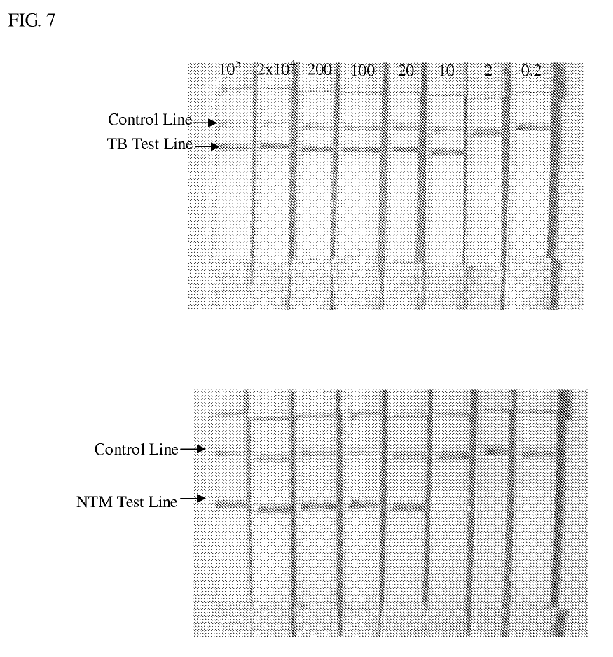

To test the sensitivity of TB/NTM strip, TB/NTM Duplex PCR and TB/NTM Nested Duplex PCR were used to amplify the DNA. The result showed that the limitation of detecting TB and NTM was 20000 copies by TB/NTM Duplex PCR; the limitation of detecting TB was 10 copies and the limitation of detecting NTM was 20 copies by TB/NTM Nested Duplex PCR (FIG. 6). The chromogenic result of the strip consisted with the electrophoresis result. The limitation of detecting TB was 10 copies and the limitation of detecting NTM was 20 copies by TB/NTM Nested Duplex PCR (FIG. 7).

### Example 5

### The Specificity and Sensitivity of TB/NTM Strip for Clinical Samples

In order to confirm the practical specificity and sensitivity of the test strip, totally clinical samples were collected and tested using TB/NTM test strip or conventional culture test for comparison. The result was as follows:

| Clinical Sample | 1 | 2 | 3 | 4 | 5 | 6 | 7 | 8 | 9 |
|---|---|---|---|---|---|---|---|---|---|
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 10 | 11 | 12 | 13 | 14 | 15 | 16 | 17 | 18 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 19 | 20 | 21 | 22 | 23 | 24 | 25 | 26 | 27 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 28 | 29 | 30 | 31 | 32 | 33 | 34 | 35 | 36 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 37 | 38 | 39 | 40 | 41 | 42 | 43 | 44 | 45 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 46 | 47 | 48 | 49 | 50 | 51 | 52 | 53 | 54 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 55 | 56 | 57 | 58 | 59 | 60 | 61 | 62 | 63 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 64 | 65 | 66 | 67 | 68 | 69 | 70 | 71 | 72 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 73 | 74 | 75 | 76 | 77 | 78 | 79 | 80 | 81 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 82 | 83 | 84 | 85 | 86 | 87 | 88 | 89 | 90 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 91 | 92 | 93 | 94 | 95 | 96 | 97 | 98 | 99 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 100 | 101 | 102 | 103 | 104 | 105 | 106 | 107 | 108 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 109 | 110 | 111 | 112 | 113 | 114 | 115 | 116 | 117 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 118 | 119 | 120 | 121 | 122 | 123 | 124 | 125 | 126 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 127 | 128 | 129 | 130 | 131 | 132 | 133 | 134 | 135 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 136 | 137 | 138 | 139 | 140 | 141 | 142 | 143 | 144 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 145 | 146 | 147 | 148 | 149 | 150 | 151 | 152 | 153 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 154 | 155 | 156 | 157 | 158 | 159 | 160 | 161 | 162 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 163 | 164 | 165 | 166 | 167 | 168 | 169 | 170 | 171 |
| Result of Culture Test | TB | TB | TB | TB | TB | TB | TB | TB | TB |
| Result of TB/NTM Test Strip | TB | TB | TB | TB | TB | TB | NTM | TB | TB |
| Clinical Sample | 172 | 173 | 174 | 175 | 176 | 177 | 178 | 179 | 180 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 181 | 182 | 183 | 184 | 185 | 186 | 187 | 188 | 189 |
| Result of Culture Test | NTM | NTM | TB | NTM | NTM | NTM | NTM | TB | NTM |
| Result of TB/NTM Test Strip | NTM | NTM | TB | NTM | NTM | NTM | NTM | TB | NTM |
| Clinical Sample | 190 | 191 | 192 | 193 | 194 | 195 | 196 | 197 | 198 |
| Result of Culture Test | TB | TB | TB | TB | TB | TB | TB | NTM | NTM |
| Result of TB/NTM Test Strip | TB | TB | TB | TB | TB | TB | TB | NTM | NTM |
| Clinical Sample | 199 | 200 | 201 | 202 | 203 | 204 | 205 | 206 | 207 |
| Result of Culture Test | NTM | NTM | NTM | NTM | NTM | NTM | NTM | NTM | NTM |
| Result of TB/NTM Test Strip | NTM | NTM | NTM | NTM | NTM | NTM | NTM | NTM | NTM |
| Clinical Sample | 208 | 209 | 210 | 211 | 212 | 213 | 214 | 215 | 216 |
| Result of Culture Test | NTM | NTM | NTM | TB | TB | TB | NTM | TB | TB |
| Result of TB/NTM Test Strip | NTM | NTM | NTM | TB | TB | TB | NTM | TB | TB |
| Clinical Sample | 217 | 218 | 219 | 220 | 221 | 222 | 223 | 224 | 225 |
| Result of Culture Test | TB | TB | TB | TB | TB | TB | TB | TB | TB |
| Result of TB/NTM Test Strip | TB | TB | TB | TB | TB | TB | TB | TB | TB |
| Clinical Sample | 226 | 227 | 228 | 229 | 230 | 231 | 232 | 233 | 234 |
| Result of Culture Test | TB | NTM | NTM | NTM | NTM | NTM | NTM | NTM | NTM |
| Result of TB/NTM Test Strip | TB | NTM | NTM | NTM | NTM | NTM | NTM | NTM | NTM |
| Clinical Sample | 235 | 236 | 237 | 238 | 239 | | | | |
| Result of Culture Test | NTM | NTM | NTM | NTM | NTM | | | | |
| Result of TB/NTM Test Strip | NTM | NTM | NTM | NTM | NTM | | | | |

There was only one (sample 169) out of 239 clinical samples tested by the TB/NTM test strip did not match to the result tested by culture. Thus, the sensitivity of the TB/NTM test strip of TB detection was 97% and the specificity of it was 100%, while the sensitivity of the TB/NTM test strip of NTM detection was 100% and the specificity of it was 97%.

While the invention has been described and exemplified in sufficient detail for those skilled in this art to make and use it, various alternatives, modifications, and improvements should be apparent without departing from the spirit and scope of the invention.

What is claimed is:

1. A strip for detecting nucleotide amplification products of Mycobacterium Tuberculosis (TB) and Non-tuberculous Mycobacterium (NTM), comprising: (a) an Avidin-Gold release region and (b) test bands, wherein the test band is a TB test band or a NTM test band.

2. The strip of claim 1, which further comprises a control band.

3. The strip of claim 1, wherein said nucleotide amplification products is DNA amplified from Polymerase Chain Reaction (PCR) using labeled primers.

4. The strip of claim 3, wherein said labeled primers is labeled with Biotin in one strand and an immunogenic molecule in the other.

5. The strip of claim 4, wherein said immunogenic molecule is Digoxigenin (DIG) or Fluorescein (FITC).

6. The strip of claim 1, wherein said TB test band is covered with antibody which binds to labeled TB DNA.

7. The strip of claim 6, wherein said TB test band is covered with anti-DIG antibody.

8. The strip of claim 1, wherein said NTM test band is covered with antibody which binds to labeled NTM DNA.

9. The strip of claim 8, wherein said NTM test band is covered with anti-FITC antibody.

10. The strip of claim 2, wherein said control band is covered with BSA-conjugated Biotin.

11. A method for detecting TB and NTM, comprising the following steps: (a) amplifying sample DNA with labeled primers; (b) mixing the amplified DNA product with running buffer; (c) dipping the test strip into the mixture; and (d) allowing the mixture to move towards the reaction region of the strip.

12. The method of claim 10, wherein said labeled primers is labeled with biotin in one strand and an immunogenic molecule in the other.

13. The method of claim 11, wherein said immunogenic molecule is DIG or FITC.

14. The method of claim 10, wherein said test strip is a strip comprising: (a) an Avidin-Gold release region and (b) test bands, wherein the test band is a TB test band or a NTM test band.
